(19) United States
(12) Patent Application Publication
(10) Pub. No.: US 2008/0146524 A1
(43) Pub. Date: Jun. 19, 2008

(54) Selective inhibitors of neurotensin degrading enzymes

(75) Inventors: Pieter Smid, Weesp (NL); Roelof W. Feenstra, Weesp (NL); Cornelis G. Kruse, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Solvay Pharmaceuticals B.V.

(21) Appl. No.: 12/000,273

(22) Filed: Dec. 11, 2007

Related U.S. Application Data
(60) Provisional application No. 60/874,711, filed on Dec. 14, 2006.

(51) Int. Cl. A61K 31/675; A61P 1/00; A61P 25/18; A61P 25/24; A61P 25/28; A61P 9/12; C07F 9/572; C12N 9/99

(52) U.S. Cl. 514/91; 435/184; 548/413

(57) ABSTRACT

Embodiments of this invention relate to compounds that are selective inhibitors of neurotensin degrading enzymes, to pharmaceutical compositions containing these compounds, to methods for preparing these compounds, methods for preparing novel intermediates useful for the synthesis of these compounds, and methods for preparing compositions containing these compounds. The invention also relates to the use of such compounds and compositions for regulating blood pressure or gastric emptying, or treating Parkinson's disease, anxiety, depression, or psychosis. The compounds have the formula (1)

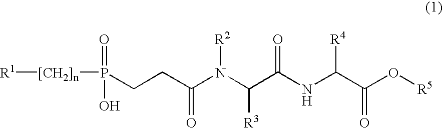

wherein the symbols have the meanings given in the specification.

This application claims the benefit of priority of U.S. Provisional Application No. 60/874,711, filed on Dec. 14, 2006, the disclosure of which is incorporated herein by reference.

| INDEX | page |
|---|---|
| Title of the invention | 1 |
| Index | 2 |
| Description of the Invention | 6 |
| Definitions | 13 |
| Abbreviations | 22 |
| Example 1: Analytical methods | 24 |
| Example 2: General aspects of syntheses | 28 |
| Example 3: Syntheses of intermediates | 29 |
| Example 4: Syntheses of specific compounds | 39 |
| Example 5: Formulations used in animal studies | 43 |
| Example 6: Pharmacological test results | 44 |
| Example 7: Chemical stability data | 45 |
| Example 8. Pharmaceutical preparations | 46 |
| Bibliography | 50 |
| Claims | 52 |

This invention relates to the fields of pharmaceutical and organic chemistry, and compounds that are selective inhibitors of neurotensin degrading enzymes, intermediates for synthesizing these compounds, methods for preparing these compounds, pharmaceutical compositions containing these compounds, and methods for preparing such compositions.

Because zinc metalloproteases metabolize proteins and peptides, they are involved in important physiological functions, and can be the origin of various pathologies. In the central nervous system (CNS), certain zinc endopeptidases (24-11, 24-15 and 24-16) are involved in the deterioration or maturation of neuropeptides. In the cardiovascular system, endothelin conversion enzymes play an essential role in regulating arterial pressure. Collagenase, elastase, gelatinase and stromelysine are zinc metalloproteases associated with ageing illnesses, and the development of cancerous metastases. In certain cases such metalloproteases have been identified as being closely associated with the virulence of certain microorganisms (botulism and tetanus toxines, cholera hemagglutine, Pseudomonas aeruginosa, and peridontal diseases due to collagenolytic bacteria).

Metalloprotease inhibitors can block degradation of numerous peptides in humans (somatostatine, bradykinine, angiotensin, neurotensin, substance P, dynorphine, VIP), thereby potentiating the effects of these peptides. Use of these inhibitors provide significant therapeutic applications involving these peptides and their degradation by endopeptidases 24-15 and 24-16 (Barelli, 1992, Vincent, 1995).

Endopeptidase 24-15 was recently implicated in Alzheimer's disease, and in the maturation stages of ras proteins, which are key proteins in the development of numerous forms of cancer. It should be noted that for similar products, namely phosphorus pseudopeptidases, tests in dogs have demonstrated that in very low concentrations these molecules effectively inhibit degradation of neurotensin (Barelli, 1994).

Neurotensin degrading enzymes are endopeptidases belonging to the family of metallopeptidases containing zinc. These neurotensin degrading enzymes have the property of inactivating certain neuromodulators such as neurotensin, thereby diminishing their pharmacological effects.

It is known that certain dipeptides such as Pro-IIe are able to inhibit endopeptidase 24.16 (Dauch, 1991^{a}). This inhibitor, however, has a Kᵢ of 90 μM, making it impossible to use in vivo, given its solubility. Moreover, the compound does not inhibit endopeptidase 24.15 at concentrations as high as 5 mM. A compound inhibiting endopeptidase 24.15 with a Kᵢ of 16 nM was described in Orlowski, 1988, and shown to also inhibit endopeptidase 24.16, albeit at 1 μM in Dauch, 1991^{b}.

Different groups have developed a rational approach for synthesizing metalloprotease inhibitors. This is based on a fundamental property of these enzymes, namely the presence in their active site of a zinc atom participating in the catalysis of the hydrolysis of the peptide bond. In global terms, this strategy involves synthesizing peptide analogs of substrates of such proteases, in which a peptide bond (C(O)═NH) is replaced by a chemical group having on the one hand good structural and electronic analogies with the peptide bond in the transition state, and on the other hand being able to strongly interact with the zinc atom present in the active site of said proteases.

Thus far, use has been made of phosphonamide (—P(O₂H)—NH), phosphone (—P(O₂H)—O) or phosphine (—P(O₂H)—CH₂) groups. The similarity of these inhibitors with substrates in the transition state generally gives these molecules exceptional affinities. The introduction of a phosphonamide bond into substrates was described in FR-A-2 654 430 and has proved to be very effective for arriving at powerful inhibitors of certain zinc proteases. However, the chemical stability of the phosphonamide bond is highly dependent on the amino acid sequences surrounding the bond and unfortunately, in certain sequences, there is a very rapid hydrolysis of the phosphonamide bond.

Use of a phosphonate-type bond has been described in Kaplan, 1991 and has made it possible to obtain, in the specific case of the carboxypeptidase A, the most powerful synthetic inhibitor reported for an enzyme thus far (inhibition constant Kᵢ=10⁻⁵ M).

Inhibitors containing a phosphine bond have been described in FR-A-2 676 059. These compounds, which resemble some of the compounds of the present invention, have proved to be very effective in the case of bacterial collagenases. N-[1-[3-[hydroxyl-(2-phenyl-ethyl)phosphinyl]1-oxopropyl]-L-prolyl]-L-norleucine, for example, these compounds have proved to be a potent inhibitor of Corynebacterium rathayii collagenase (Yiotakis, 1994)

EP 0 565 450 describes inhibitors containing a phosphonamide bond, which are very effective with respect to the endopeptidase 24.15, and are also very good inhibitors of endopeptidase 24.16. Structurally related peptide derivatives in which a peptide bond has been replaced by a phosphine bond were disclosed in EP 0 725 075. They were shown to be selective inhibitors of the endopeptidase 24-15, while being inactive with respect to other zinc peptidases such as endopeptidase 24-16. Other structurally related phosphinate based inhibitors of matrix metallo-proteases were disclosed in WO 98/03516.

Thus, any peptide containing a phosphine bond is a potential inhibitor of different proteases belonging to the family of zinc metalloproteases. However, apart from interactions of the phosphine bond with the zinc atom of the active site, the affinity of the peptide is also dependent on interactions between amino acids on either side of the phosphine unit, and different subsites of the active site of the protease.

One object of the present invention is to develop compounds that are potent and selective inhibitors of endopeptidases 24-15 and 24-16.

### DESCRIPTION OF THE INVENTION

It was found that compounds of the general formula (1) are selective inhibitors of neurotensin degrading enzymes. The invention relates to compounds of formula (1):

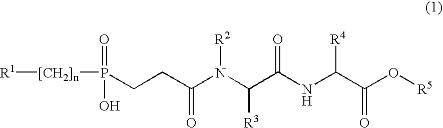

and tautomers, stereoisomers, N-oxides, isotopically-labeled analogues, or pharmacologically acceptable salts, hydrates or solvates of any of the foregoing wherein:
R¹ is chosen from a monocyclic aryl group, a monocyclic heteroaryl group, a bicyclic aryl group, and a bicyclic heteroaryl group, which groups are optionally substituted;
when R¹ is a monocyclic aryl group or a monocyclic heteroaryl group, n is 3, 4 or 5, and when R¹ is a bicyclic aryl group or a bicyclic heteroaryl group, n is 1, 2, 3, 4 or 5;
R² is a hydrogen atom or a (C₁₋₃)alkyl group, or R² and R³, together with the atoms to which they are attached, may form a five or six membered ring which may contain a sulfur atom;
R³ is chosen from a hydrogen atom, a branched or unbranched (C₁₋₈)alkyl group, and an optionally substituted benzyl group;
R⁴ is chosen from a hydrogen atom, a branched or unbranched (C₁₋₈)alkyl group, and an optionally substituted benzyl group;
R⁵ is chosen from a hydrogen atom, a methyl group, an ethyl group, a methoxymethyl group and an ethoxymethyl group.

In some embodiments, the invention relates to a compound of formula (1) in which R¹ is an optionally substituted phenyl or naphthyl group, R² is a hydrogen atom or a methyl group, or R₂ and R₃, together with the atoms to which they are attached, may form a five-membered ring, which may contain a sulfur atom, and n, R³, R⁴ and R⁵ have the meanings as given above.

In other embodiments, the invention relates to one or more compounds of formula (1) wherein R¹ is a phenyl or naphthyl group, R² is a hydrogen atom, or R₂ and R₃, together with the atoms to which they are attached, may form a five-membered ring, which may contain a sulfur atom, R₃ is a branched or unbranched (C₁₋₄)alkyl group, R⁴ is a branched or unbranched (C₁₋₄)alkyl group, R⁵ is a hydrogen atom, and n has the meaning as given above.

A further embodiment provides a compound of formula (1′):

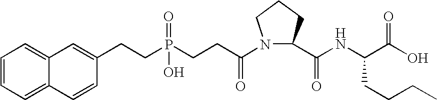

In another embodiment the invention relates to compounds of formula (2):

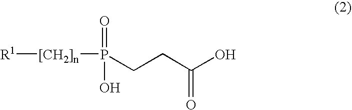

wherein:
R¹ is chosen from a monocyclic aryl group, a monocyclic heteroaryl group, a bicyclic aryl group, and a bicyclic heteroaryl group, which groups are optionally substituted,
when R¹ is a monocyclic aryl group or a monocyclic heteroaryl group, n is 3, 4 or 5, and when R¹ is a bicyclic aryl group or a bicyclic heteroaryl group, n is 1, 2, 3, 4 or 5, with the proviso that when n is 4, R¹ is not an unsubstituted phenyl group. The compound of formula (2) may be useful in the synthesis of compounds of formula (1).

The compounds of the invention are new and are selective inhibitors of neurotensin degrading enzymes. More specifically, the compounds inhibit the enzymes Thimet oligopeptidase EC 3.4.24.15, and Neurolysine EC 3.4.24.16, which break down the neuropeptide neurotensin. The compounds are active in inhibiting the above-mentioned enzymes in the range of 5.0-9.0 (pIC₅₀ values), when tested according to published methods (Dauch, 1991^{a,b}). Due to the inhibition of the neurotensin degrading activity of these enzymes the levels of endogenous neurotensin will rise, causing benificial effects in the treatment of diseases in which neurotensin levels are disturbed, such as peripheral disturbances like regulation of blood pressure and gastric emptying, neurological disturbances like Parkinson's disease, and central nervous system disturbances like anxiety, depression, psychosis and other psychotic disorders.

Other embodiments of the invention include, but are not limited to:

pharmaceutical compositions for treating, for example, a disorder or condition treatable by inhibiting neurotensin degrading enzymes, the composition comprising a compound of formula (1), and a pharmaceutically acceptable carrier;

methods of treating a disorder or condition treatable by inhibiting neurotensin degrading enzymes, the method comprising administering to a mammal in need of such treating a compound of formula (1);

pharmaceutical compositions for treating, for example, a disorder or condition chosen from peripheral disturbances like regulation of blood pressure and gastric emptying, neurological disturbances like Parkinson's disease, and central nervous system disturbances like anxiety, depression, psychosis and other psychotic disorders;

methods of treating a disorder or condition chosen from the disorders listed herein, the methods comprising administering to a mammal in need of such treating a compound of formula (1);

pharmaceutical compositions for inhibiting neurotensin degrading enzymes, the compositions comprising a compound of formula (1), and a pharmaceutically acceptable carrier;

methods for inhibiting neurotensin degrading enzymes, the methods comprising administering to a patient in need of such treating a compound of formula (1); and

methods for inhibiting neurotensin degrading enzymes that comprises administering to a subject in need thereof, an effective amount of a compound of formula (1).

Still other embodiments of the invention relate to the use of a compound according to formula (1) for the manufacture of a medicament.

The invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for treating one or more of the conditions listed herein. Such other therapeutic agent(s) may be administered prior to, simultaneously with, or following the administration of the compounds of the invention.

The invention also provides compounds, pharmaceutical compositions, kits and methods for inhibiting neurotensin degrading enzymes, the method comprising administering to a patient in need of such treating a compound of formula (1).

The compounds of the invention possess neurotensin degrading enzyme inhibiting activities. The inhibiting activities of the compounds of the invention are readily demonstrated, for example, using one or more of the assays described herein, or known in the art.

The invention also provides methods of preparing the compounds of the invention and the intermediates used in those methods.

Isolation and purification of the compounds and intermediates described herein can be affected, if desired, by any suitable separation or purification procedure such as, for example, filtration, extraction, crystallization, column chromatography, thin-layer chromatography, thick-layer chromatography, preparative low or high-pressure liquid chromatography, or a combination of these procedures. Specific illustrations of suitable separation and isolation procedures can be taken from the preparations and examples. However, other equivalent separation or isolation procedures could, of course, also be used.

The compounds of the present invention may contain one or more asymmetric centers and can thus occur as racemates and racemic mixtures, single enantiomers, diastereomeric mixtures and individual diastereomers.

Depending on the nature of the various substituents, the molecule can have additional asymmetric centers. Each such asymmetric center will independently produce two optical isomers. All of the possible optical isomers and diastereomers, in mixtures and as pure or partially purified compounds, belong to this invention. The present invention comprehends all such isomeric forms of these compounds. Formula (1) shows the structure of the class of compounds without preferred stereochemistry. The independent syntheses of these diastereomers, or their chromatographic separations, may be achieved as known in the art by appropriate modification of the methodology disclosed therein. Their absolute stereochemistry may be determined by the X-ray crystallography of crystalline products or crystalline intermediates, which are derivatized, if necessary, with a reagent containing an asymmetric center of known absolute configuration. Racemic mixtures of the compounds can be separated into the individual enantiomers by methods well-known in the art, such as the coupling of a racemic mixture of compounds to an enantiomerically pure compound to form a diastereomeric mixture, followed by separation of the individual diastereomers by standard methods, such as fractional crystallization or chromatography. The coupling often consists of the formation of salts using an enantiomerically pure acid or base, for example (−)-di-p-toluoyl-D-tartaric acid and/or (+)-di-p-toluoyl-L-tartaric acid. The diasteromeric derivatives may then be converted to the pure enantiomers by cleavage of the added chiral residue. The racemic mixture of the compounds can also be separated directly by chromatographic methods utilizing chiral stationary phases, which are methods well-known in the art. Alternatively, any enantiomer of a compound may be obtained by stereoselective synthesis using optically pure starting materials or reagents of known configuration by methods well-known in the art.

Cis and trans isomers of the compound of formula (1) are within the scope of the invention, and this also applies to tautomers of the compounds of formula (1).

Some of the crystalline forms for the compounds may exist as polymorphs, which are also within the scope of the invention. In addition, some of the compounds may form solvates with water (i.e., hydrates) or common organic solvents. Such solvates also fall within the scope of this invention.

A compound of formula (1) isotopically-labeled to be detectable by PET or SPECT also falls within the scope of the invention. The same applies to compounds of formula (1) labeled with [¹³C]-, [¹⁴C]-, [³H]-, [¹⁸F]-, [¹²⁵I]- or other isotopic suitable for receptor binding or metabolism studies.

The compounds of the invention may also be used as reagents or standards in the biochemical study of neurological function, dysfunction, and disease.

### DEFINITIONS

General terms used in the description of compounds herein disclosed bear their usual meanings. The term alkyl as used herein denotes a univalent saturated branched or straight hydrocarbon chain. Unless otherwise stated, such chains can contain from 1 to 18 carbon atoms. Representative of such alkyl groups are methyl, ethyl, propyl, isopropyl, butyl, isobutyl, sec-butyl, tert-butyl, pentyl, isopentyl, neopentyl, tert-pentyl, hexyl, isohexyl, heptyl, octyl, nonyl, decyl, undecyl, dodecyl, tridecyl, tetradecyl, pentadecyl, hexadecyl, heptadecyl, octadecyl, and the like. When qualified ‘lower’, the alkyl group will contain from 1 to 6 carbon atoms. The same carbon content applies to the parent term ‘alkane’, and to derivative terms such as ‘alkoxy’. The carbon content of various hydrocarbon containing moieties is indicated by a prefix designating the minimum and maximum number of carbon atoms in the moiety, i.e., the prefix Cₓ-C_{y} defines the number of carbon atoms present from the integer “x” to the integer “y” inclusive. ‘Alkyl(C₁₋₃)’, for example, means methyl, ethyl, n-propyl or isopropyl, and ‘alkyl(C₁₋₄)’ means ‘methyl, ethyl, n-propyl, isopropyl, n-butyl, 2-butyl, isobutyl or 2-methyl-n-propyl’. The term ‘alkenyl’ denotes straight or branched hydrocarbon radicals having one or more carbon-carbon double bonds, such as vinyl, allyl, butenyl, etc., and for example represents (C₂₋₄)alkenyl. In ‘alkynyl= groups the straight or branched hydrocarbon radicals have one or more carbon-carbon triple bonds, such as ethynyl, propargyl, 1-butynyl, 2-butynyl, etc., and for example represent (C₂₋₄)alkynyl. Unless otherwise stated, ‘alkenyl’ and ‘alkynyl chains can contain from 1 to 18 carbon atoms.

The term ‘acyl” means alkyl(C₁₋₃) carbonyl, arylcarbonyl or aryl-alkyl(C₁₋₃)carbonyl. ‘Aryl’ embraces monocyclic or fused bicyclic aromatic or hetero-aromatic groups, including but not limited to furyl, thienyl, pyrrolyl, oxazolyl, thiazolyl, imidazolyl, imidazo[2,1-b][1,3]thiazolyl, pyrazolyl, isoxazolyl, isothiazolyl, pyridyl, pyridazinyl, pyrimidinyl, pyrazinyl, 1,3,5-triazinyl, phenyl, indazolyl, indolyl, indolizinyl, isoindolyl, benzo[b]furanyl, 1,2,3,4-tetrahydro-naphtyl, 1,2,3,4-tetrahydroisoquinolinyl, indanyl, indenyl, benzo[b]thienyl, 2,3-dihydro-1,4-benzodioxin-5-yl, benzimidazolyl, benzothiazolyl, benzo[1,2,5]thia-diazolyl, purinyl, quinolinyl, isoquinolinyl, phtalazinyl, quinazolinyl, quinoxalinyl, 1,8-naphthyridinyl, naphthyl, pteridinyl or azulenyl. ‘Halo’ or ‘Halogen’ means chloro, fluoro, bromo or iodo; ‘hetero’ as in ‘heteroalkyl, heteroaromatic’ etc. means containing one or more N, O, or S atoms; ‘heteroalkyl’ includes alkyl groups with heteroatoms in any position, thus including N-bound, O-bound, or S-bound alkyl groups.

The term “substituted” means that the specified group or moiety bears one or more substituents, where any group may carry multiple substituents, and a variety of possible substituents is provided, the substituents are independently selected, and need not to be the same. The term “unsubstituted” means that the specified group bears no substituents. With reference to substituents, the term “independently” means that when more than one of such substituents are possible, they may be the same or different from each other.

‘Optionally substituted’ means that a group may or may not be further substituted by one or more groups selected from C₁₋₈ alkyl, C₁₋₈ alkenyl, C₁₋₈ alkynyl, aryl, fluoro, chloro, bromo, hydroxyl, C₁₋₈alkyloxy, C₁₋₈alkenyloxy, aryloxy, acyloxy, amino, C₁₋₈ alkylamino, dialkyl(C₁₋₈)amino, arylamino, thio, C₁₋₈alkylthio, arylthio, cyano, oxo, nitro, acyl, amido, C₁₋₈ alkylamido, dialkyl(C₁₋₈)amido, carboxyl, or two optional substituents may together with the carbon atoms to which they are attached form a 5- or 6-membered aromatic or non-aromatic ring containing 0, 1 or 2 heteroatoms selected from nitrogen, oxygen or sulfur. Optional substituents may themselves bear additional optional substituents. Some optional substituents include C₁₋₃ alkyl such as for example, methyl, ethyl, and trifluoromethyl, fluoro, chloro, bromo, hydroxyl, C₁₋₃ alkyloxy such as for example methoxy, ethoxy and trifluoromethoxy, and amino.

The terms “oxy”, “thio” and “carbo” as used herein as part of another group respectively refer to an oxygen atom, a sulfur atom and a carbonyl (C═O) group, serving as a linker between two groups, such as for instance hydroxyl, oxyalkyl, thioalkyl, carboxyalkyl, etc. The term “amino” as used herein alone, or as part of another group, refers to a nitrogen atom that may be either terminal, or a linker between two other groups, wherein the group may be a primary, secondary or tertiary (two hydrogen atoms bonded to the nitrogen atom, one hydrogen atom bonded to the nitrogen atom and no hydrogen atoms bonded to the nitrogen atom, respectively) amine.

To provide a more concise description, the terms ‘compound’ or ‘compounds’ include tautomers, stereoisomers, N-oxides, isotopically-labeled analogues, or pharmacologically acceptable salts, hydrates or solvates, also when not explicitly mentioned.

N-oxides of the compounds mentioned above belong to the invention. Tertiary amines may or may not give rise to N-oxide metabolites. The extent to what N-oxidation takes place varies from trace amounts to a near quantitative conversion. N-oxides may be more active than their corresponding tertiary amines, or less active. Whilst N-oxides can easily be reduced to their corresponding tertiary amines by chemical means, in the human body this happens to varying degrees. Some N-oxides undergo nearly quantitative reductive conversion to the corresponding tertiary amines, in other cases conversion is a mere trace reaction, or even completely absent (Bickel, 1969).

Any compound metabolized in vivo to provide the bioactive agent (i.e., the compound of formula (1)) is a prodrug within the scope and spirit of the application. Prodrugs are therapeutic agents, inactive per se but transformed into one or more active metabolites. Thus, in the methods of treatment of the present invention, the term “administering” shall encompass treating the various disorders described with the compound specifically disclosed, or with a compound that not specifically disclosed, but that converts to the specified compound in vivo after administration to the patient. Prodrugs are bioreversible derivatives of drug molecules used to overcome some barriers to the utility of the parent drug molecule. These barriers include, but are not limited to, solubility, permeability, stability, presystemic metabolism and targeting limitations (Bundgaard, 1985; King, 1994; Stella, 2004; Ettmayer, 2004; Järvinen, 2005). Prodrugs, i.e. compounds that when administered to humans by any known route, are metabolised to compounds having formula (1), belong to the invention. In particular this relates to compounds with primary or secondary amino or hydroxy groups. Such compounds can be reacted with organic acids to yield compounds having formula (1) wherein an additional group is present that is easily removed after administration, for instance, but not limited to amidine, enamine, a Mannich base, a hydroxyl-methylene derivative, an O-(acyloxymethylene carbamate) derivative, carbamate, ester, amide or enaminone.

‘Crystal form’ refers to various solid forms of the same compound, for example polymorphs, solvates and amorphous forms. ‘Polymorphs’ are crystal structures in which a compound can crystallize in different crystal packing arrangements, all of which have the same elemental composition. Polymorphism is a frequently occurring phenomenon, affected by several crystallization conditions such as temperature, level of supersaturation, the presence of impurities, polarity of solvent, rate of cooling. Different polymorphs usually have different X-ray diffraction patterns, solid state NMR spectra, infrared or Raman spectra, melting points, density, hardness, crystal shape, optical and electrical properties, stability, and solubility. Recrystallization solvent, rate of crystallization, storage temperature, and other factors may cause one crystal form to dominate. ‘Solvates’ are generally a crystal form that contains either stoichiometric or non-stoichiometric amounts of a solvent. Often, during the process of crystallization some compounds have a tendency to trap a fixed molar ratio of solvent molecules in the crystalline solid state, thus forming a solvate. When the solvate is water, ‘hydrates’ may be formed. The compound of formula (1) and pharmaceutically acceptable salts thereof may exist in the form of a hydrate or a solvate, and such a hydrate and solvate are also encompassed in the present invention. Examples thereof include 1/10 hydrate, ¼ hydrate, ½ hydrate, monohydrate, dihydrochloride ½ hydrate, dihydrochloride dihydrate, dihydrochloride 3/2 hydrate, and the like. ‘Amorphous’ forms are noncrystalline materials with no long range order, and generally do not give a distinctive powder X-ray diffraction pattern. Crystal forms in general have been described by Byrn (1995) and Martin (1995).

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

Throughout the description and the claims of this specification the word “comprise” and variations of the word, such as “comprising” and “comprises” is not intended to exclude other additives, components, integers or steps.

While it may be possible for the compounds of formula (1) to be administered as the raw chemical, it is also possible to present them as a ‘pharmaceutical composition’. According to a further aspect, the present invention provides a pharmaceutical composition comprising a compound of formula (1), or a pharmaceutically acceptable salt or solvate thereof, together with one or more pharmaceutically acceptable carriers thereof, and optionally one or more other therapeutic ingredients. The carrier(s) must be ‘acceptable’ in the sense of being compatible with the other ingredients of the formulation and not deleterious to the recipient thereof. The term “composition” as used herein encompasses a product comprising specified ingredients in predetermined amounts or proportions, as well as any product that results, directly or indirectly, from combining specified ingredients in specified amounts. In relation to pharmaceutical compositions, this term encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. The pharmaceutical composition includes enough of the active object compound to produce the desired effect upon the progress or condition of diseases. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

Within the context of this application, the term ‘combination preparation’ comprises both true combinations, meaning a compound of formula (1) and other medicaments physically combined in one preparation such as a tablet or injection fluid, as well as ‘kit-of-parts’, comprising a compound of formula (1) and another medicament in separate dosage forms, together with instructions for use, optionally with further means for facilitating compliance with the administration of the component compounds, e.g., label or drawings. With true combinations, the pharmacotherapy by definition is simultaneous. The contents of ‘kit-of-parts’, can be administered either simultaneously or at different time intervals. Therapy being either concomitant or sequential will be dependant on the characteristics of the other medicaments used, characteristics like onset and duration of action, plasma levels, clearance, etc., as well as on the disease, its stage, and characteristics of the individual patient.

“Dose” as used herein refers to the potency of the compounds of the invention as inhibitors of neurotensin degrading enzymes, which was determined as described herein. From the potency measured for a given compound of formula (1), one can estimate a theoretical lowest effective dose. At a concentration of the compound equal to twice the measured inhibition constant, nearly 100% of the neurotensin degrading enzymes likely will be occupied by the compound. By converting that concentration to mg of compound per kg of patient one obtains a theoretical lowest effective dose, assuming ideal bioavailability. Pharmacokinetic, pharmacodynamic, and other considera-tions may alter the dose actually administered to a higher or lower value. The typical daily dose of the active ingredients varies within a wide range and will depend on various factors such as the relevant indication, the route of administration, the age, weight and sex of the patient, and may be determined by a physician. In general, total daily dose administration to a patient in single or individual doses, may be in amounts, for example, from 0.001 to 10 mg/kg body weight daily, and more usually from 0.01 to 1,000 mg per day, of total active ingredients. Such dosages will be administered to a patient in need of treatment from one to three times each day, or as often as needed for efficacy; and for periods of at least two months, more typically for at least six months, or chronically.

The term “therapeutically effective amount” as used herein refers to an amount of a therapeutic agent to treat a condition treatable by administrating a composition of the invention. That amount is the amount sufficient to exhibit a detectable therapeutic or ameliorative response in a tissue system, animal or human. The effect may include, for example, treating the conditions listed herein. The precise effective amount for a subject will depend upon the subject's size and health, the nature and extent of the condition being treated, recommendations of the treating physician (researcher, veterinarian, medical doctor or other clinician), and the therapeutics, or combination of therapeutics, selected for administration. Thus, it is not useful to specify an exact effective amount in advance. The term “pharmaceutically acceptable salt” refers to those salts that are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. They can be prepared in situ when finally isolating and purifying the compounds of the invention, or separately by reacting them with pharmaceutically acceptable non-toxic bases or acids, including inorganic or organic bases and inorganic or organic acids (Berge, 1977). The ‘free base’ form may be regenerated by contacting the salt with a base or acid, and isolating the parent compound in the conventional matter. The parent form of the compound differs from the various salt forms in certain physical properties, such as solubility in polar solvents, but otherwise the salts are equivalent to the parent form of the compound for the purposes of the present invention. ‘Complex’ refers to a complex of the compound of the invention, e.g. formula (1), complexed with a metal ion, where at least one metal atom is chelated or sequestered. Complexes are prepared by methods well known in the art (Dwyer, 1964).

The term “treatment” as used herein refers to any treatment of a mammalian, for example human condition or disease, and includes: (1) inhibiting the disease or condition, i.e., arresting its development, (2) relieving the disease or condition, i.e., causing the condition to regress, or (3) stopping the symptoms of the disease. The term ‘inhibit’ includes its generally accepted meaning which includes prohibiting, preventing, restraining, alleviating, ameliorating, and slowing, stopping or reversing progression, severity, or a resultant symptom. As such, the present method includes both medical therapeutic and/or prophylactic administration, as appropriate. As used herein, the term “medical therapy” is intended to include prophylactic, diagnostic and therapeutic regimens carried out in vivo or ex vivo on humans or other mammals. ‘Mammals” include animals of economic importance such as bovine, ovine, and porcine animals, especially those that produce meat, as well as domestic animals, sports animals, zoo animals, and humans. The term “subject” as used herein, refers to an animal, for example a mammal, or a human, who has been the object of treatment, observation or experiment.

### ABBREVIATIONS

ACN acetonitrile
AIBN 2,2′-azobis-(2-methylpropionitrile)
API-ES atmospheric pressure ionization—electron spray
CNS central nervous system
CUR curtain gas
DCM dichloromethane
DIPEA N,N-diisopropylethylamine
DMF N,N′-dimethylformamide
DMSO dimethylsulfoxide
EP entrance potential
ESI-MS electron spray ionization mass spectrometry
EtOAc ethylacetate
EtOH ethanol
Et₂O diethyl ether
Et₃N triethyl amine
FMoc N-alpha-(9-fluorenylmethyloxycarbonyl)-
FP focusing potential
g gram(s)
h hour(s)
HATU 2-(1H-7-Azabenzotriazol-1-yl)-1,1,3,3-tetramethyl-uronium-Hexafluorophosphate
HBTU O-Benzotriazole-N,N,N′,N′-tetramethyl-uronium-hexafluorophosphate
HOAt 1-hydroxy-7-azabenzotriazole
HPLC high performance liquid chromatography
IS ionspray voltage
LC liquid chromatography
MeOH methanol
mg milligram(s)
min minute(s)
ml milliliter(s)
m.p. melting point c.q. melting range
MS mass spectrometry
NEB nebulizer gas
NMP N-methylpyrrolidone
PA petroleum aether
PET positron emission tomography
R_{f} retention factor (thin layer chromatography)
Rₜ retention time (LC/MS)
SPECT single photon emission computed tomography
TEM temperature
TLC thin layer chromatography
TFA trifluoroacetic acid p0 VIP vasoactive intestinal polypeptide

### EXAMPLE 1

### Analytical Methods

Nuclear magnetic resonance spectra (¹H NMR and ¹³C NMR, APT) were determined in the indicated solvent using a Bruker ARX 400 (¹H: 400 MHz, ¹³C: 100 MHz) at 300 K, unless indicated otherwise. ¹⁹F NMR and ¹³C NMR experiments were carried out on a Varian Inova 500 spectrometer operating at 11.74 T (499.9 MHz for ¹H; 125.7 MHz for ¹³C; 50.7 Mhz, 470.4 MHz for ¹⁹F) using a 5 mm SW probe. The spectra were determined in deuterated chloroform or dichloromethane obtained from Cambridge Isotope Laboratories Ltd. Chemical shifts (6) are given in ppm downfield from tetramethylsilane (¹H, ¹³C) or CCl₃F (¹⁹F). Coupling constants J are given in Hz. Peakshapes in the NMR spectra are indicated with the symbols ‘q’ (quartet), ‘dq’ (double quartet), ‘t’ (triplet), ‘dt’ (double triplet), ‘bt’ (broad triplet), ‘d’ (doublet), ‘dd’ (double doublet), ), ‘bd’ (broad doublet), ‘s’ (singlet), ‘bs’ (broad singlet) and ‘m’ (multiplet). NH and OH signals were identified after mixing the sample with a drop of D₂O.

Flash chromatography refers to purification using the indicated eluent and silica gel (Acros: 0.030-0.075 mm or Merck silica gel 60: 0.040-0.063 mm).

Column chromatography: silica gel 60 (0.063-0.200 mm, Merck).

Melting points were recorded on a Büchi B-545 melting point apparatus.

Mass spectra were recorded on a Micromass QTOF-2 instrument with MassLynx application software for acquisition and reconstruction of the data. Exact mass measurement was done of the quasimolecular ion [M+H]⁺.

All reactions involving moisture sensitive compounds or conditions were carried out under an anhydrous nitrogen atmosphere.

Reactions were monitored by using thin-layer chromatography (TLC) on silica coated plastic sheets (Merck precoated silica gel 60 F254) with the indicated eluent. Spots were visualized by UV light (254 nm) or I₂.

Dichloromethane (phosphorous pentoxide and calciumhydride), tetrahydro-furan (sodium/benzophenone ketyl) and light petroleum (60-80) were distilled freshly prior to use. All other commercially available chemicals were used without further purification.

Analytical HPLC was performed on a C18 column (Inertsil ODS-3, particle size 3 mm; 4.6 mm 50 mm) using the following elution gradient: linear gradient of 5% to 95% aqueous CH₃CN containing 0.04% HCO₂H over 12 min, then 95% aqueous CH₃CN containing 0.04% HCO₂H for 4 min at 2.0 ml min⁻¹. Products were detected at λ=254 nm or 225 nm.

### Liquid Chromatography-Mass Spectrometrry (LC-MS)

The LC-MS system consists of 2 Perkin elmer series 200 micro pumps. The pumps are connected to each other by a 50 μl tee mixer, connected to a Gilson 215 auto sampler. The method is as follows:

| step | total time | flow (μl/min) | A(%) | B(%) |
|---|---|---|---|---|
| 0 | 0 | 2000 | 95 | 5 |
| 1 | 1.8 | 2000 | 0 | 100 |
| 2 | 2.5 | 2000 | 0 | 100 |
| 3 | 2.7 | 2000 | 95 | 5 |
| 4 | 3.0 | 2000 | 95 | 5 |

A = 100% Water with 0.025% HCOOH and 10 mmol NH₄HCOO pH = ±3
B = 100% ACN with 0.025% HCOOH

The auto sampler has a 2 μl injection loop. The auto sampler is connected to a Waters Atlantis C18 30*4.6 mm column with 3 μm particles. The column is thermo stated in a Perkin Elmer series 200 column oven at 40° C. The column is connected to a Perkin Elmer series 200 UV meter with a 2.7 μl flowcel. The wavelength is set to 254 nm. The UV meter is connected to a Sciex API 150EX mass spectrometer. The mass spectrometer has the following parameters:

Scan range:150-900 a.m.u.; polarity: positive; scan mode: profile; resolution Q1: UNIT; step size: 0.10 a.m.u.; time per scan: 0.500 sec; NEB: 10; CUR: 10 IS: 5200; TEM: 325; DF: 30; FP: 225 and EP: 10.

The light scattering detector is connected to the Sciex API 150. The light scattering detector is a Sedere Sedex 55 operating at 50° C. and 3 bar N₂. The complete system is controlled by a G3 powermac.

### Determination of Chemical Stability

Compound 36 and its phosphinamide analog phosphodiepril (FR 2 654 430, synthesized as disclosed therein) were stored separately in glass vials. At different time intervals samples were taken and analyzed by LC-MS. Samples were dissolved in DMSO (0.1 mg/ml) and diluted by a factor 100 in the first LC eluent (A). At fixed time points an amount of 100 μl was taken from the formulations. These time points were 0, 3, 72, and 240 hours. All samples were measured in the positive mode, with an ESI source on the LC-MS.

Eluents were composed of water (H₂O), acetonitril (ACN), methanol (MeOH) and ammonium-acetate (NH₄Ac). The eluent is mixed out of two different bottles with two different compositions.
Eluent A consists of H₂O/ACN/MeOH 800/100/100+0.77 g/l NH₄Ac.
Eluent B consists of H₂O/ACN/MeOH 100/800/100+0.77 g/l NH₄Ac.

The gradient in the pump was set to:

| Time (min) | % A | % B |
|---|---|---|
| 0 | 100 | 0 |
| 3.6 | 0 | 100 |
| 7.2 | 0 | 100 |
| 8.5 | 100 | 0 |

Columns: Chromsep Guard Column SS 10×2 mm (CP28141) and Inertsil 5 ODS-3 100×3.0 mm (CP22234). Column temperature: 25° C.

Injection: well plate temperature: 25° C.
Injection volume: 20 μl
Splitter (post column): 1:4
Run time: 9.50 minutes.

Detection MS-MS:ESI (pos/neg) spray 3.0 kV
Fragmentor 70
Gain 2.0
Dwell 700 msec
Nebulizer pressure 42 psig
Drying gas temperature 325° C.
Capillary temperature 325° C.

### EXAMPLE 2

### General Aspects of Syntheses

The synthesis of compounds having formula (1) is outlined in Scheme 1. Compounds can be prepared by both solid phase and solution phase chemistry. Examples of both routes are described. The amino acids X1 and X2 can be naturally occurring or chemically synthesized, having either the D or L configuration. Amino acids bound to Wang resin can either be bought, prepared by methods well known to those skilled in the art of solid phase chemistry.

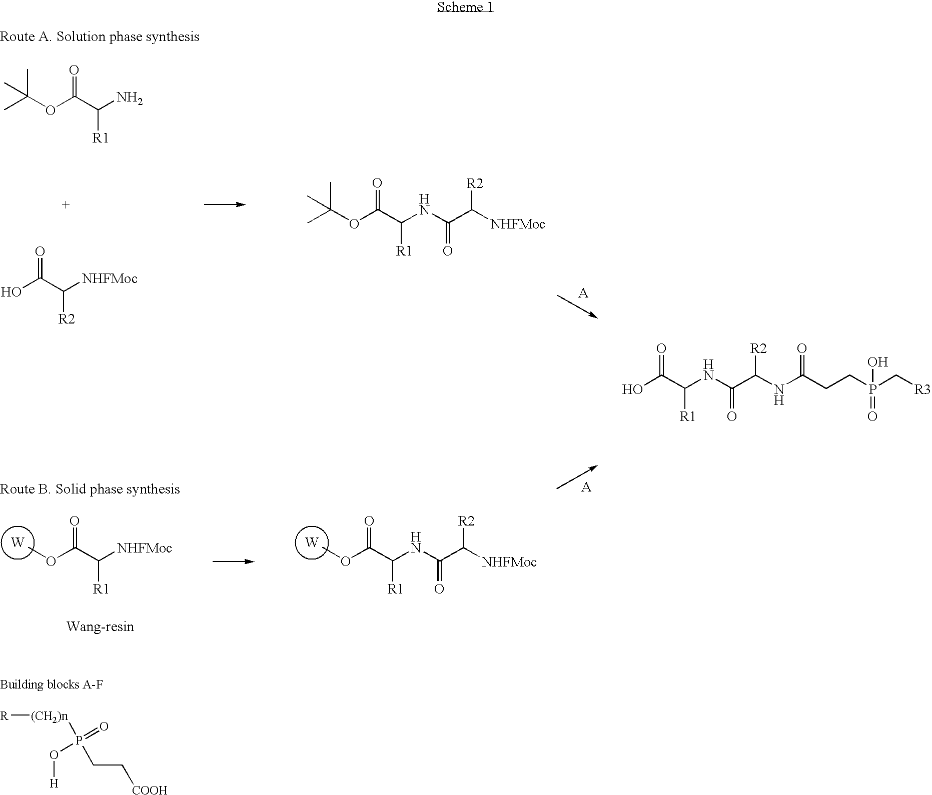

Methods for making phosphinic acid intermediates have been disclosed in U.S. Pat. Nos. 4,594,199 and 4,602,092.

The selection of the particular synthetic procedures depends on factors known to those skilled in the art such as the compatibility of functional groups with the reagents used, choice of solid phase material, the possibility to use protecting groups, catalysts, activating and coupling reagents and the ultimate structural features present in the final compound being prepared.

Pharmaceutically acceptable salts may be obtained using standard procedures well known in the art, for example, by mixing a compound of the present invention with a suitable acid, for instance an inorganic acid or an organic acid.

### EXAMPLE 3

### Syntheses of Intermediates

Synthesis of intermediate carboxylic acid derivatives A is outlined in Scheme 2.

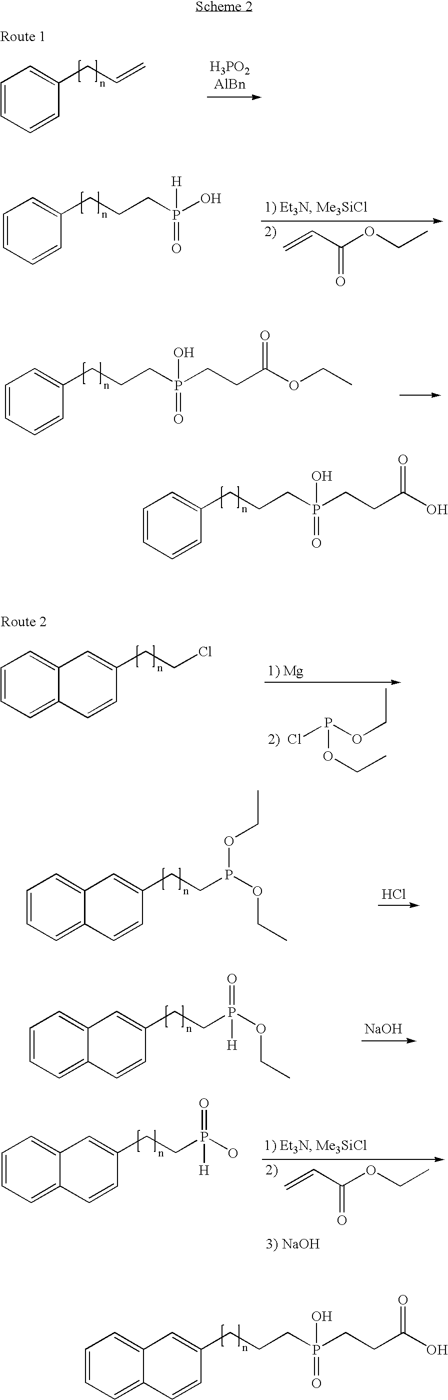

3-phenyl-propyl-phosphinic acid (Compound A1; see Karanewsky, 1988). To a solution of hypophosphorous acid (50 wt % in water, 228 mmol, 23.6 ml) in 250 ml ethanol was added commercially available allyl benzene (10 ml, 76.3 mmol) and AIBN (2,2′-azobis(2-methylpropio-nitrile), 1 g). The mixture was heated to reflux for 6 hours under a nitrogen atmosphere, after which another portion of AIBN (1 g) was added. The mixture was subsequently refluxed for 18 hours. The solution was concentrated in vacuo. The resulting oil was cooled to 0° C. and 400 ml 2N NaOH was added. The solution was washed with diethyl ether (3×300 ml). The water layer was acidified with 3N HCl and extracted with ethyl acetate (3×300 ml). The combined organic layers were washed with a saturated solution of NaCl (400 ml), dried on magnesium sulfate and concentrated in vacuo to yield compound A1 (10 g, 72%) as an oil. TLC (i-PrOH/NH₄OH/H₂O, 85/10/5, v/v/v, R_{f} 0.25). ¹H NMR (CDCl₃): 10.5 (s, 1H, P—OH); 6.38 (t), 7.74 (t) (1H, PH); 7.1-7.3 (m, 5H, H-arom); 2.7 (t, 2H, CH₂); 1.68-1.96 (m, 4H, 2×CH₂). ESI-MS [M−H] 183.

3-[Hydroxy-(3-phenyl-propyl)-phosphinoyl]-propionic acid ethyl ester (Compound A2). 3-phenyl-propyl-phosphinic acid (10 g, 54.6 mmol) was dissolved in dry dichloromethane (200 ml). To the cooled solution (0-5° C.) was slowly added (15 minutes) triethyl amine (17.1 ml, 122.6 mmol) and subsequently over a period of 30 minutes trimethyl silyl chloride (15.6 ml, 122.6 mmol) was added. Stirring was continued for 90 minutes after which time acrylic acid ethyl ester (7.7 ml, 60 mmol) was added over a period of 15 minutes. The mixture was stirred for 16 hours at room temperature. The solution was acidified with 1 N hydrochloric acid (200 ml). The water layer was extracted with dichloromethane and the combined organic layers were washed with water (2×200 ml), filtered over a WAF-filter and the filtrate was concentrated in vacuo to give crude A2 as an oil. The mixture was separated by flashchromatography (100% DCM to DCM/MeOH/NH₄OH, 84/15/1, v/v/v) to give pure compound A2 (13.5 g, 87%) as an oil. TLC (DCM/MeOH/NH₄OH, 84/15/1, v/v/v, Rf 0.15). ¹H NMR (CDCl₃): 9.5 (s, 1H, P—OH); 7.1-7.3 (5H, H-arom); 4.1 (q, 2H, CH₂O); 2.5-2.7 (t, 4H, 2×CH₂); 1.68-2.1 (m, 6H, 3×CH₂); 1.2 (t, 3H, CH₃).

3-[Hydroxy-(3-phenyl-propyl)-phosphinoyl]-propionic acid (Compound A3) 3-[Hydroxy-(3-phenyl-propyl)-phosphinoyl]-propionic acid ethyl ester (13.5 g, 47 mmol) was dissolved in EtOH (300 ml) and 2N NaOH (65 ml) was added. The solution was stirred for 70 hours and subsequently concentrated in vacuo. The resulting oil was cooled in an ice bath, 1 N HCl (150 ml) was added and the mixture extracted with EtOAc (3×250 ml). The combined organic layers were washed with a saturated solution of NaCl (400 ml), dried on magnesium sulfate and concentrated in vacuo to yield compound A3 as a white solid. The solid was stirred in Et₂O (100 ml) and filtered. The resulting white powder was dried in vacuo to furnish pure A3 (8.92 g, 74%). TLC (EtOAc/MeOH/AcOH, 50/45/5, v/v/v, R_{f} 0.25). ¹H NMR (CDCl₃): 9.3 (s, 1H, P—OH); 7.1-7.3 (5H, H-arom); 2.64 (t, 2H, CH₂); 2.5-2.6 (m, 2H, CH₂); 1.6-2.1 (m, 6H, 3×CH₂). ESI-MS [M−H] 254.9.

3-phenyl-butyl-phosphinic acid (Compound B1). 3-phenyl-butyl-phosphinic acid was prepared as described for A1 yielding compound B1 as an oil (9.3 g, 94%). TLC (i-PrOH/NH₄OH/H2O, 85/10/5, v/v/v, Rf 0.3). 1H NMR (CDCl₃): 10.0 (s, 1 H, P—OH); 5.7 (bt), 8.4 (bt) (1H, PH); 7.1-7.3 (5H, H-arom); 2.7 (t, 2H, CH₂); 1.6-1.9 (m, 6H, 3×CH₂). ESI-MS [M−H] 196.9.

3-[Hydroxy-(3-phenyl-butyl)-phosphinoyl]-propionic acid ethyl ester. (Compound B2). 3-[Hydroxy-(3-phenyl-butyl)-phosphinoyl]-propionic acid ethyl ester was prepared as described for A2 to give pure compound B2 (12.6 g, 90%) as an oil. TLC (DCM/MeOH/NH₄OH, 84/15/1, v/v/v, Rf 0.2). 1H NMR (CDCl₃): 8.5 (bs, 1H, P—OH); 7.0-7.3 (5H, H-arom); 4.1 (q, 2H, CH₂O); 2.4-2.6 (m, 4H, 2×CH₂); 1.68-2.1 (m, 8H, 4×CH₂); 1.2 (t, 3H, CH₃).

3-[Hydroxy-(3-phenyl-butyl)-phosphinoyl]-propionic acid (Compound B3). 3-[Hydroxy-(3-phenyl-butyl)-phosphinoyl]-propionic acid was prepared as described for A3 to yield compound B3 (9 g, 79%). TLC (EtOAc/MeOH/AcOH 50/45/5, v/v/v, R_{f} 0.3). ¹H NMR (DMSO): 7.1-7.3 (5H, H-arom); 2.64 (t, 2H, CH₂); 2.32-2.4 (m, 2H, CH₂); 1.5-1.85 (m, 8H, 4×CH₂). ESI M−H 268.9

2-naphthalen-2-yl-ethylphosphonous acid diethyl ester (Compound C1).

The procedure of the first step was executed according a method described in WO 97/048409 and EP 0 071 544. To a mixture of magnesium powder (1.28 g, 53 mmol) in dry Et₂O (3 ml) was added some Iodine crystals and the mixture was heated to reflux. 2-(2-Chloroethyl)-naphtalene (10.1 g, 53 mmol) in 50 ml dry Et₂O was placed in a dropping funnel. The solution was slowly added to the magnesium suspension while maintaining reflux conditions. Refluxing was continued for 3 hours, after which time the mixture was cooled in an ice bath. The mixture was filtered under a nitrogen atmosphere. The filtrate was added over a period of 90 minutes and under a nitrogen atmosphere to a solution of diethylchlorophophite (7.6 ml, 53 mmol) in Et₂O (50 ml) at a temperature of 0° C. A white slurry was formed and the mixture was stirred for an additional 16 hours at room temperature under a blanket of nitrogen. The reaction mixture was filtered and the filtrate was evaporated in vacuo to give crude compound C1 (12 g) which was used in the next step without further purification.

(2-Naphthalen-2-yl-ethyl)-phosphinic acid ethyl ester (Compound C2). Crude C1 (12 g) was suspended in water (8 ml) and 0.3 ml of concentrated HCl. The reaction mixture heated up because of the exothermic reaction. Stirring was continued for 2 hours after which time the reaction mixture was extracted with EtOAc (3×50 ml). The combined organic layers were washed with a saturated solution of NaCl (2×90 ml), dried on magnesium sulfate and concentrated in vacuo to yield C2 as an oil (10.9 g) which was used in the next step without further purification.

(2-Naphthalen-2-yl-ethyl)-phosphinic acid (Compound C3). A suspension of crude C2 (10.9 g) in 2N NaOH (50 ml) was stirred for 1 hr at room temperature. The mixture was washed with Et₂O (3×40 ml) and the water layer was acidified with concentrated HCl to pH 1. The acidic water layer was extracted with EtOAc (3×50 ml) and the combined EtOAc layers was washed with a saturated solution of NaCl (2×90 ml), dried on magnesium sulfate and concentrated in vacuo to yield compound C3 as an oil (5.3 g, 46%) which was used in the next step without further purification. TLC (DCM/MeOH/NH₄OH, 84/15/1, v/v/v, R_{f} 0.1). ¹H NMR (CDCl₃): 11.0 (bs, 1H, P—OH); 5.8 (bt), 8.5 (bt) (1H, PH); 7.2-7.9 (m, 7H, H-arom); 3.1 (m, 2H, CH₂); 2.0-2.3 (m, 2H, CH₂). ESI-MS [M−H] 218.9.

3-[Hydroxy-(2-Naphthalen-2-yl-ethyl)-phosphinoyl]proprionic acid ethyl ester (Compound C4). (2-Naphtalen-2-yl-ethyl)-phosphinic acid (C3, 5.3 g, 24 mmol) was dissolved in dry DCM (90 ml) and cooled to 0° C. in an ice bath. To the cooled solution was added Et3N (7.5 ml, 54 mmol) and trimethylsilyl chloride (6.87 ml, 54 mmol) and the mixture was stirred for 1 hr, after which time acrylic acid ethyl ester (3.4 ml, 26.6 mmol) was added over a period of 15 minutes. The mixture was stirred for 2 hours at room temperature. The solution was cooled to 0° C. in an ice bath and acidified with 1N hydrochloric acid (90 ml). The water layer was extracted with dichloromethane (3×70 ml) and the combined organic layers were washed with water (2×100 ml), filtered over a WAF-filter and the filtrate was concentrated in vacuo to give crude C4 as an oil. The mixture was separated by flash chromatography (100% DCM to DCM/MeOH/NH₄OH, 84/15/1, v/v/v) to give pure C4 (8.1 g, 99%) as an oil. TLC (DCM/MeOH/NH₄OH, 84/15/1, v/v/v, R_{f} 0.15). ¹H NMR (CDCl₃): 7.9 (bs, 1 H, P—OH); 7.1-7.7 (7H, H-arom); 3.9-4.0 (q, 2H, CH₂O); 3.0 (m, 2H, CH₂); 2.5 (m, 2H, CH₂); 1.8-2.0 (m, 4H, 2×CH₂); 1.1 (t, 3H, CH₃).

3-[Hydroxy-(2-Naphthalen-2-yl-ethyl)-phosphinoyl]proprionic acid (Compound C5). 3-[Hydroxy-(2-Naphtalen-2-yl-ethyl)-phosphinoyl]proprionic acid ethyl ester (C4, 8.1 g, 25.3 mmol) was dissolved in EtOH (160 ml). To the solution was added 2N NaOH (35 ml) and the mixture was stirred for 3 hours at room temperature and subsequently concentrated in vacuo. The resulting oil was cooled in an ice bath, 1 N HCl (80 ml) was added and the mixture extracted with EtOAc/MeOH, 3/1, v/v (3×150 ml). The combined organic layers were washed with a saturated solution of NaCl (400 ml), dried on magnesium sulfate and concentrated in vacuo to yield C5 as a white solid. The solid was stirred in Et₂O (100 ml) and filtered. The resulting white powder was dried in vacuo to furnish C5 (5.97 g, 81%). TLC (EtOAc/MeOH/AcOH, 50/45/5, v/v/v, Rf 0.25). Melting point: 165-167° C.; NMR (DMSO): 7.4-7.7 (7H, H-arom); 3.0 (m, 2H, CH₂); 2.5 (m, 2H, CH₂); 1.8-2.1 (m, 4H, 2×CH₂).

2-(naphthalen-1-yl)-ethylphosphonous acid diethyl ester (Compound D1). Compound D1 was prepared following the same procedure as described for C1. Crude D1 (14 g) was used in the next step without further purification.

2-(Naphthalen-1-yl)-ethyl-phosphinic acid ethyl ester (Compound D2).

D2 was prepared following the same procedure as for C2. Compound D2 was isolated as an oil (13 g) which was used in the next step without further purification.

2-(Naphthalen-1-yl)-ethyl-phosphinic acid (Compound D3). Compound D3 was prepared following the same procedure as described for compound C3. Compound D3 was isolated as an oil (7.1 g) which was used in the next step without further purification. TLC (DCM/MeOH/NH₄OH, 84/15/1, v/v/v, R_{f} 0.1). ¹H NMR (CDCl₃): 9.5 (bs, 1H, P—OH); 6.5 (bt), 7.9 (bt) (1H, PH); 7.3-8.0 (7H, H-arom); 3.4 (m, 2H, CH₂); 2.2-2.3 (m, 2H, CH₂).

3-[Hydroxy-(2-[Naphthalen-1-yl]-ethyl)-phosphinoyl]proprionic acid ethyl ester (Compound D4). D4 was prepared following the same procedure as described for compound C4. Compound D4 was isolated as an oil (8.1 g, 79%); TLC (DCM/MeOH/NH₄OH, 84/15/1, v/v/v, Rf 0.15). 1H NMR (CDCl₃): 7.1-7.9 (8H, P—OH and H-arom); 3.9-4.0 (q, 2H, CH₂O); 3.25 (m, 2H, CH₂); 2.5 (m, 2H, CH₂); 1.8-2.0 (m, 4H, 2×CH₂); 1.1 (t, 3H, CH₃).

3-[Hydroxy-(2-Naphthalen-1-yl-ethyl)-phosphinoyl]proprionic acid (Compound D5). D5 was prepared following the same procedure as described for compound C5. Compound D5 was isolated as a white solid (6.6 g). TLC (EtOAc/MeOH/AcOH, 50/45/5, v/v/v, Rf 0.3). Melting point: 136-139° C.; 1H NMR (DMSO): 7.4-8.1 (7H, H-arom); 3.3 (m, 2H, CH₂); 2.5 (m, 2H, CH₂); 1.9-2.1 (m, 4H, 2×CH₂). ESI-MS, [M−H] 290.9.

(Naphthalen-1-yl)-methylphosphonous acid diethyl ester (Compound E1).

E1 was prepared following the same procedure as described for C1. Compound E1 was isolated as an oil (11 g), which was used in the next step without further purification.

(Naphthalen-1-yl-methyl)-phosphinic acid ethyl ester (Compound E2). Compound E2 was prepared following the same procedure as described for C2. Compound E2 was isolated as an oil (10.2 g), which was used in the next step without further purification.

(Naphthalen-1-yl-methyl)-phosphinic acid (Compound E3). Compound E3 was prepared following the same procedure as described for C3. Compound E3 was isolated as an oil (3.8 g, 37%). TLC (DCM/MeOH/NH₄OH, 84/15/1, v/v/v, Rf 0.1). 1H NMR (CDCl₃): 9.0 (bs, 1H, P—OH); 5.6 (bt), 8.4 (bt) (1H, PH); 7.3-8.0 (7H, H-arom); 3.5-3.6 (dd, 2H, P—CH₂).

3-[Hydroxy-(Naphthalen-1-yl-methyl)-phosphinoyl]proprionic acid ethyl ester (Compound E4). E4 was prepared following the same procedure as described for C4. Compound E4 was isolated as an oil (6.6 g, 83%); TLC (DCM/MeOH/NH₄OH, 84/15/1, v/v/v, Rf 0.15). 1H NMR (CDCl₃): 7.2-8.1 (7H, H-arom); 6.7 (bs, 1H, P—OH); 4.0 (q, 2H, CH₂O); 3.3-3.4 (dd, 2H, P—CH₂); 2.4-2.5 (m, 2H, CH₂); 1.7-1.9 (m, 2H, CH₂); 1.1 (t, 3H, CH₃).

3-[Hydroxy-(Naphthalen-1-yl-methyl)-phosphinoyl]proprionic acid (E5). Compound E5 was prepared following the same procedure as described for compound C5. Compound E5 was isolated as a white solid (5.3 g, 89%). TLC (EtOAc/MeOH/AcOH, 50/45/5, v/v/v, Rf 0.3). Melting point: 187-189° C.; NMR (DMSO): 7.4-8.2 (7H, H-arom); 3.6 (d, 2H, P—CH₂); 2.4 (m, 2H, CH₂); 1.8-1.9 (m, 2H, CH₂).

Naphthalen-2-yl-methylphosphonous acid diethyl ester (Compound F1). Compound F1 was prepared following the same procedure as described for C1. Compound F1 was isolated as an oil (10.1 g), which was used in the next step without further purification.

(Naphthalen-2-yl-methyl)-phosphinic acid ethyl ester (Compound F2). F2 was prepared following the same procedure as described for compound C2. Compound F2 was isolated as an oil (7.1), which was used in the next step without further purification.

(Naphthalen-2-yl-methyl)-phosphinic acid (Compound F3). F3 was prepared following the same procedure as described for C3. Compound F3 was isolated as an oil (1.4 g, 14% yield based on naftyl methylene bromide), which was used in the next step without further purification. TLC (DCM/MeOH/NH₄OH, 84/15/1, v/v/v, Rf 0.1). 1H NMR (CDCl₃): 8.7 (bs,1H, P—OH); 5.6 (bt), 8.4 (bt) (1H, PH); 7.2-7.9 (7H, H-arom); 3.2-3.3 (dd, 2H, P—CH₂).

3-[Hydroxy-(Naphthalen-2-yl-methyl)-phosphinoyl]proprionic acid ethyl ester (Compound F4). Compound F4 was prepared following the same procedure as described for C4. Compound F4 was isolated after flash chromatography as an oil (2.7 g, 70%). TLC (DCM/MeOH/NH₄OH, 84/15/1, v/v/v, Rf 0.2). 1H NMR (CDCl₃): 7.0 (bs, 1 H, P—OH); 7.2-7.7 (7H, H-arom); 4.0 (q, 2H, CH₂O); 3.0-3.1 (dd, 2H, P-CH₂); 2.3-2.5 (m, 2H, CH₂); 1.7-1.9 (m, 2H, CH₂); 1.1 (t, 3H, CH₃).

3-[Hydroxy-(Naphthalen-2-yl-methyl)-phosphinoyl]proprionic acid (F5). F5 was prepared following the same procedure as described for C5. Compound F5 was isolated as a white solid (2.1 g, 88%). TLC (EtOAc/MeOH/AcOH, 50/45/5, v/v/v, Rf 0.3). Melting point: 210° C.; NMR (DMSO): 7.4-7.8 (7H, H-arom); 3.2-3.3 (d, 2H, CH₂); 2.4 (m, 2H, CH₂); 1.8 (m, 2H, CH₂).

### Route A:

The properly protected dipeptides can be prepared following a solution phase route. The route is described for the synthesis of (I)-Pro-(I)-Norleucine tButyl ester, but is widely applicable for the synthesis of all dipeptides disclosed.

FMoc-(I)-Pro-(I)-Norleucine tButyl ester (Compound G1). To a mixture of FMoc protected I-Proline (1.35 g, 4 mmol) and tButyl ester protected (I) norleucine (0.75 g 4 mmol) in NMP (10 ml) was added HOAt (0.54 g, 4 mmol), HBTU (1.52 g, 4 mmol) and DIPEA (0.87 ml, 5 mmol). The mixture was stirred for 16 hours at room temperature, and thereafter diluted with 5% NaHCO3 (5 ml) and extracted with EtOAc (3×75 ml). The combined organic layers were washed with a saturated solution of NaCl (100 ml), dried on magnesium sulfate and concentrated in vacuo to yield crude G1. Crude compound G1 was purified by flash column chromatography (EtOAc/PA, ½, v/v) furnishing pure G1 as a white solid (1.84 g, 91%). TLC (EtOAc/PA, ½, V/V, Rf 0.25).

(I)-Pro-(I)-Norleucine tButyl ester (Compound G2). To a solution of G1 (1.84 g, 3.64 mmol) in THF (25 ml) was added piperidine (1.45 ml). The reaction mixture was stirred for 1 hour at room temperature after which time TLC analysis showed that the reaction was complete. The mixture was concentrated in vacuo and the residue was purified by silica gel column chromatography (100% DCM to DCM/MeOH, 9/1, v/v) to give pure compound G2 (1.0 g, 97%) as an oil. TLC (DCM/MeOH, 9/1, v/v, Rf 0.25).

### Route B:

The properly protected dipeptides can also be prepared following a solid phase route. In this way the C-terminus protection of the growing peptide chain is the solid phase material, Wang resin with a loading capacity of 0.7 mmol/g. The route is described for the synthesis of (I)-Pro-(I)-Norleucine, but is widely applicable for the synthesis of all dipeptides claimed.

FMoc-(I)-Pro-(I)-Norleucine Wang-resin bound. To a mixture of FMoc protected I-Proline (1.35 g, 4 mmol) and N-terminal unprotected, C-terminal resin bound (I) norleucine (1.4 g of resin, 1 mmol) in NMP (10 ml) was added HOAt (0.54 g, 4 mmol), HBTU (1.52 g, 4 mmol) and DIPEA (0.87 ml, 5 mmol). The mixture was shaken for 2 hours at room temperature. The mixture was filtered and the resin was washed with 3×10 ml DMF, 2×10 ml MeOH, 2×10 ml DCM, 2×10 ml DMF. The resulting resin was negative in the bromophenol blue test indicating that all amines were converted to amides.

(I)-Pro-(I)-Norleucine Wang-resin bound. FMoc-(I)-Pro-(I)-Norleucine Wang-resin bound (2 g resin) was mixed in a solution of piperidine in DMF (10 ml, 20%). The mixture was mixed for 10 minutes and the resin was filtered, washed with DMF and the reaction was repeated for a second time. The mixture was filtered and the resin was washed with 3×10 ml DMF, 2×10 ml MeOH, 2×10 ml DCM, 2×10 ml DMF, and used without further preparation in the next step.

### EXAMPLE 4

### Syntheses of Specific Compounds

The specific compounds of which the synthesis is described below are intended to further illustrate the invention in more detail, and therefore are not deemed to restrict the scope of the invention in any way. Other embodiments of the invention will be apparent to those skilled in the art from consideration of the specification and practice of the invention disclosed herein. It is thus intended that the specification and examples be considered as exemplary only.

3-[Hydroxy-(3-phenyl-butyl)-phosphinoyl]-propionyl)-2-amino-(3-methyl-butyrylamino)hexcanoic acid (in codes: 3-[Hydroxy-(3-phenyl-butyl)-phosphi-noyl]-propionyl)-(I)-Val-(I)-NLeu-OH], Compound 6). To a suspension of NH₂-Val-NLeu-C(═O)—O— Wang resin (300 mg, 0.25 mmol) NMP (5 ml) was added compound B3 (0.2 g, 0.75 mmol, 0.25M in NMP). The mixture was shaken under an inert Nitrogen atmosphere. To the mixture was added a 0.5M solution of HATU in NMP (1.5 ml, 0.75 mmol) and DIPEA (0.75 mmol, 0.13 ml). The mixture was shaken overnight, after which time the mixture was filtered and the redidue resin was washed with NMP (3×10 ml). The reaction was repeated under the same conditions for 6 hours after which time a nynhydrin assay showed that the reaction was complete. The mixture was filtered and the residue resin was washed with DCM (2×10 ml), MeOH (2×10 ml), DMF (2×10 ml), DCM (2×10 ml), MeOH (2×10 ml), DMG (2×10 ml), DCM (2×10 ml). To the resin was added a cocktail of TFA/DCM/water, 70/25/5, v/v/v and the mixture was shaken for 3 hours at room temperature. The mixture was filtered and the filtrate was evaporated with water (3×30 ml) to give compound 6 as a white solid. TLC (i-PrOH/NH₄OH/H2O, 85/10/5, v/v/v, R_{f} 0.1). HPLC purity: 70%, Rₜ 7.3 min; mass spectroscopy: ESI [M−H] 481.1.

2-[(1-{Hydroxy-(2-naphtalen-2-yl-ethyl)phosphi noyl}-propionyl)-pyrrolidine-carbonyl]-amino]-hexanoic acid t-butyl ester (in codes: 2-[(1-{Hydroxy-(2-naphtalen-2-yl-ethyl)phosphinoyl}-propionyl)-(I)-Pro-(I)-NLeu t-butyl ester], Compound 36): To a solution of Pro-NLeu-O-tBu ester (G2, 0.45 g, 1.6 mmol) and compound C5 (0.47 g, 1.6 mmol) in dry DCM (25 ml) was added dicyclohexyl carbodiimide (DCC, 0.73 g, 3.5 mmol). The mixture was stirred for 16 hours under nitrogen at room temperature, whereafter TLC showed that the reaction was complete (DCM/MeOH/NH₄OH, 84/15/1, v/v/v, Rf 0.15). The reaction mixture was filtered and concentrated in vacuo. The mixture was purified by silica gel column chromatography (DCM to DCM/MeOH/NH₄OH, 84/15/1, v/v/v) to give compound 36-tBu ester as an oil (0.71 g, 80%). TLC (DCM/MeOH/NH₄OH, 84/15/1, v/v/v, R_{f} 0.15).

2-[(1-{Hydroxy-(2-naphtalen-2-yl-ethyl)phosphinoyl}-propionyl)-pyrrolidine-carbonyl]-amino]-hexanoic acid (in codes: 2-[(1-{Hydroxy-(2-naphta-len-2-yl-ethyl)phosphinoyl}-propionyl)-(I)-Pro-(I)-NLeu-OH]): To a solution of compound 36-tBu ester (0.69 g, 1.2 mmol) in DCM (11 ml) was added TFA (8 ml) and the mixture was stirred for 16 hours at room temperature. The mixture was diluted with toluene and the mixture was concentrated in vacuo. Co-evaporation was repeated two times with toluene and two times with can to yield compound 36 as a white solid. The compound was stirred overnight in Et₂O to furnish 36 as an amorphous compound (0.6 g, 97%). TLC (i-PrOH/NH₄OH/H2O, 85/10/5, v/v/v, Rf 0.1). 1H NMR (CDCl₃): 7.0-7.8 (9H, H-arom, P—OH, NH); 4.3-4.5 (2×m, 2H, CH-Pro, CH—NLeu); 3.4 (m, 2H, Pro-CH₂); 3.0 (m, 2H, naf-CH₂); 2.5 (m, 2H, C(═O)CH₂); 1.6-2.1 (m, 10H, 5× CH₂); 1.3 (m, 4H, 2×NLeu-CH₂); 0.8 (bt, 3H, CH₃—NLeu). HPLC purity: 91%, Rₜ 7.6 min; mass spectroscopy: ESI [M−H] 501.1.

Analogous to the syntheses of the compounds 6 and 36, detailed above, the compounds 1-5, 7-35 and 37-39 (all listed in the table below) were accomplished. Under the heading “S” the specific synthetic route (either A or B, as outlined above) is given. The column headed HPLC gives the purity (%) as well as the retention time (Rₜ). In the next column (TLC) R_{f}-values are given, chromatography being performed using Merck's plates precoated with silica gel 60 F254, and i-PrOH/NH₄OH/H₂O, 85/10/5, v/v/v as eluent. In the last column, the ‘√’ symbol indicates that observed ESI-[M−H]-values are identical with, or very close to, calculated values.

| | |
|---|---|
| | (1) |
| 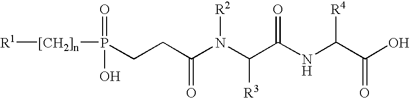 | |

| C | R₁ | n | R₂ | R₃ | R₄ | S | HPLC | TLC | MS |
|---|---|---|---|---|---|---|---|---|---|
| 1 | phenyl | 3 | H | i-propyl | n-butyl | B | 81%, Rt 7.0 | | √ |
| 2 | phenyl | 3 | H | i-propyl | benzyl | B | 64%, Rt 5.5 | | √ |
| 3 | phenyl | 3 | H | 2-butyl | i-propyl | B | 80%, Rt 6.8 | | √ |
| 4 | phenyl | 3 | H | 2-butyl | i-butyl | A | 95%, Rt 5.7 | 0.1 | |
| 5 | phenyl | 3 | H | benzyl | ethyl | B | 50%, Rt 6.8 | | √ |
| 6 | phenyl | 4 | H | i-propyl | n-butyl | B | 67%, Rt 7.3 | | √ |
| 7 | phenyl | 4 | H | i-propyl | benzyl | B | 65%, Rt 7.4 | | √ |
| 8 | phenyl | 4 | H | 2-butyl | i-propyl | B | 88%, Rt 5.8 | | √ |
| 9 | phenyl | 4 | H | 2-butyl | i-butyl | A | 95%, Rt 5.9 | 0.1 | |
| 10 | phenyl | 4 | H | benzyl | ethyl | B | 50%, Rt 7.2 | | √ |
| 11 | phenyl | 4 | methyl | methyl | n-butyl | A | | 0.15 | |
| 1213 | phenylphenyl | 34 | 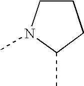 | | n-butyln-butyl | BB | 74%, Rt 6.778%, Rt 7.0 | | √√ |
| 1415 | Phenylphenyl | 34 | 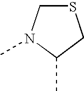 | | n-propyln-propyl | BB | 80%, Rt 6.680%, Rt 7.0 | | √√ |
| 16 | α-naphthyl | 1 | H | i-propyl | n-butyl | B | | | |
| 17 | β-naphthyl | 1 | H | i-propyl | n-butyl | B | | | |
| 18 | α-naphthyl | 1 | H | i-propyl | benzyl | B | | | |
| 19 | β-naphthyl | 1 | H | i-propyl | benzyl | B | 70% | | √ |
| 20 | α-naphthyl | 1 | H | 2-butyl | i-propyl | B | | | |
| 21 | β-naphthyl | 1 | H | 2-butyl | i-propyl | B | 80% | | |
| 22 | α-naphthyl | 1 | H | 2-butyl | i-butyl | A | | 0.1 | √ |
| 23 | β-naphthyl | 1 | H | 2-butyl | i-butyl | A | | 0.1 | √ |
| 24 | α-naphthyl | 1 | H | benzyl | ethyl | B | | | |
| 25 | β-naphthyl | 1 | H | benzyl | ethyl | B | 50% | | |
| 26 | α-naphthyl | 2 | H | i-propyl | n-butyl | B | | | |
| 27 | α-naphthyl | 2 | H | i-propyl | benzyl | B | | | |
| 28 | α-naphthyl | 2 | H | 2-butyl | i-propyl | B | | | |
| 29 | β-naphthyl | 2 | H | 2-butyl | i-butyl | A | | 0.1 | √ |
| 30 | α-naphthyl | 2 | H | 2-butyl | i-butyl | A | | 0.1 | √ |
| 31 | α-naphthyl | 2 | H | benzyl | ethyl | B | | | |
| 32 | β-naphthyl | 2 | methyl | methyl | n-butyl | A | | 0.15 | |

| | | | | | | | | |
|---|---|---|---|---|---|---|---|---|
| 33343536 | α-naphthylβ-naphthylα-naphthylβ-naphthyl | 1122 | 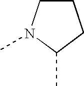 | n-butyln-butyln-butyln-butyl | BBBA | 91%, Rt 7.6 | 0.1 | √ |
| 373839 | α-naphthylβ-naphthylα-naphthyl | 112 | 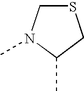 | n-propyln-propyln-propyl | BBB | 60% | | |

Of the compounds in the table, all amino acids formed by R₂, R₃ and R₄ are L-amino acids

### EXAMPLE 5

### Formulations Used in Animal Studies

For oral (p.o.) administration: to the desired quantity (0.5-5 mg) of the solid compound 36 in a glass tube, some glass beads were added and the solid was milled by vortexing for 2 minutes. After addition of 1 ml of a solution of 1% methylcellulose in water and 2% (v/v) of Poloxamer 188 (Lutrol F68), the compound was suspended by vortexing for 10 minutes. The pH was adjusted to 7 with a few drops of aqueous NaOH (0.1N). Remaining particles in the suspension were further suspended by using an ultrasonic bath.

For intraperitoneal (i.p.) administration: to the desired quantity (0.5-15 mg) of the solid compound 36 in a glass tube, some glass beads were added and the solid was milled by vortexing for 2 minutes. After addition of 1 ml of a solution of 1% methylcellulose and 5% mannitol in water, the compound was suspended by vortexing for 10 minutes. Finally the pH was adjusted to 7.

### EXAMPLE 6

### Pharmacological Test Results

The compounds of the invention are selective inhibitors of Thimet oligopeptidase EC 3.4.24.15 and Neurolysine EC 3.4.24.16, which break down neurotensin. pIC₅₀ values of the compounds range from 5.0-9.0, when tested according to published methods (Dauch, 1991^{a,b}). Representative data are given the table below.

enzyme inhibition

| | EC 3.4.24.15 | EC 3.4.24.16 |
|---|---|---|
| Cmp | pIC₅₀ | pIC₅₀ |
| 6 | 6.8 | 6.7 |
| 7 | 6.2 | 6.1 |
| 8 | 5.8 | 5.4 |
| 9 | 6.3 | 5.6 |
| 11 | 6.2 | 6.1 |
| 12 | 6.3 | 6.3 |
| 13 | 7.6 | 8.2 |
| 14 | 5.1 | 5.7 |
| 15 | 6.8 | 7.8 |
| 9 | 6.3 | 5.6 |
| 11 | 6.2 | 6.1 |
| 12 | 6.3 | 6.3 |
| 13 | 7.6 | 8.2 |
| 14 | 5.1 | 5.7 |
| 15 | 6.8 | 7.8 |
| 25 | 5.7 | 5.9 |
| 26 | 6.5 | 6.3 |
| 27 | 6.2 | 6.1 |
| 28 | 6.3 | 5.8 |
| 29 | 7.0 | 6.6 |
| 30 | 5.8 | 5.2 |
| 32 | 7.0 | |
| 35 | 7.2 | 7.4 |
| 36 | 8.9 | >7.5 |
| 39 | 6.9 | 7.2 |

### EXAMPLE 7

### Stability Data

Compound 36 and its phosphinamide analog phosphodiepril (FR 2 654 430, synthesized as disclosed therein) were stored separately in glass vials. At different time intervals samples were taken and analyzed by LC-MS.

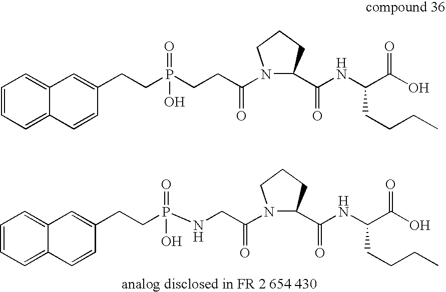

When calculating the relative stability it was assumed that the compounds were completely dissolved during formulation. Therefore, the relative stability is 100% at the beginning (0 hour measurement). The following time measurements are recalculated from that measurement.

| Time (hr) | phosphodiepril | Compound 36 |
|---|---|---|
| 0 | 100 | 100 |
| 3 | 92 | 101 |
| 72 | 87 | 103 |
| 240 | 82 | 100 |

Compound 36 remained stable over 10 days (240 hr), while phosphodiepril gradually degraded. Thus, even in its pure form, the N-analog is not stable. Due to the rapid hydrolysis of the phosphonamide bond, enzyme inhibition activity of this compound can only be determined with difficulty. In vivo experiments invariably produced negative results.

### EXAMPLE 8

### Pharmaceutical Preparations

For clinical use, compounds of formula (1) are formulated into pharmaceutical compositions that are important and novel embodiments of the invention because they contain the compounds, more particularly the specific compounds disclosed herein. Types of pharmaceutical compositions that may be used include, but are not limited to, tablets, chewable tablets, capsules (including microcapsules), solutions, parenteral solutions, ointments (creams and gels), suppositories, suspensions, and other types disclosed herein, or apparent to a person skilled in the art from the specification and general knowledge in the art. The active ingredeient for instance, may also be in the form of an inclusion complex in cyclodextrins, their ethers or their esters. The compositions are used for oral, intravenous, subcutaneous, tracheal, bronchial, intranasal, pulmonary, transdermal, buccal, rectal, parenteral or other ways to administer. The pharmaceutical formulation contains at least one compound of formula (1) in admixture with a pharmaceutically acceptable adjuvant, diluent and/or carrier. The total amount of active ingredients suitably is in the range of from about 0.1% (w/w) to about 95% (w/w) of the formulation, from about 0.5% to 50% (w/w), or from about 1% to 25% (w/w).

The compounds of the invention can be brought into forms suitable for administration by means of usual processes using auxiliary substances such as liquid or solid, powdered ingredients, such as the pharmaceutically customary liquid or solid fillers and extenders, solvents, emulsifiers, lubricants, flavorings, colorings and/or buffer substances. Frequently used auxiliary substances include magnesium carbonate, titanium dioxide, lactose, saccharose, sorbitol, mannitol and other sugars or sugar alcohols, talc, lactoprotein, gelatin, starch, amylopectin, cellulose and its derivatives, animal and vegetable oils such as fish liver oil, sunflower, groundnut or sesame oil, polyethylene glycol and solvents such as, for example, sterile water and mono- or polyhydric alcohols such as glycerol, as well as with disintegrating agents and lubricating agents such as magnesium stearate, calcium stearate, sodium stearyl fumarate and polyethylene glycol waxes. The mixture may then be processed into granules or pressed into tablets. A tablet is prepared using the ingredients below:

| Ingredient | Quantity (mg/tablet) |
|---|---|
| COMPOUND No. 36 | 10 |
| Cellulose, microcrystalline | 200 |
| Silicon dioxide, fumed | 10 |
| Stearic acid | 10 |
| Total | 230 |

The components are blended and compressed to form tablets each weighing 230 mg.

The active ingredients may be separately premixed with the other non-active ingredients, before being mixed to form a formulation. The active ingredients may also be mixed with each other, before being mixed with the non-active ingredients to form a formulation.

Soft gelatin capsules may be prepared with capsules containing a mixture of the active ingredients of the invention, vegetable oil, fat, or other suitable vehicle for soft gelatin capsules. Hard gelatin capsules may contain granules of the active ingredients. Hard gelatin capsules may also contain the active ingredients together with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories that contain the active substance mixed with a neutral fat base; (ii) in the form of a gelatin rectal capsule that contains the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration.

Liquid preparations may be prepared in the form of syrups, elixirs, concentrated drops or suspensions, e.g., solutions or suspensions containing the active ingredients and the remainder consisting, for example, of sugar or sugar alcohols and a mixture of ethanol, water, glycerol, propylene glycol and polyethylene glycol. If desired, such liquid preparations may contain coloring agents, flavoring agents, preservatives, saccharine and carboxymethyl cellulose or other thickening agents. Liquid preparations may also be prepared in the form of a dry powder, reconstituted with a suitable solvent prior to use. Solutions for parenteral administration may be prepared as a solution of a formulation of the invention in a pharmaceutically acceptable solvent. These solutions may also contain stabilizing ingredients, preservatives and/or buffering ingredients. Solutions for parenteral administration may also be prepared as a dry preparation, reconstituted with a suitable solvent before use.

Also provided according to the present invention are formulations and ‘kits of parts’ comprising one or more containers filled with one or more of the ingredients of a pharmaceutical composition of the invention, for use in medical therapy. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration. The use of formulations of the present invention in the manufacture of medicaments for use in treating a condition in which inhibition of neurotensin degrading enzymes is required or desired, and methods of medical treatment or comprising the administration of a therapeutically effective total amount of at least one compound of formula (1) to a patient suffering from, or susceptible to, a condition in which inhibition of neurotensin degrading enzymes is required or desired.

By way of example and not of limitation, several pharmaceutical compositions are given, comprising active compounds for systemic use or topical application. Other compounds of the invention or combinations thereof, may be used in place of (or in addition to) said compounds. The concentration of the active ingredient may be varied over a wide range as discussed herein. The amounts and types of ingredients that may be included are well known in the art.

### BIBLIOGRAPHY

Barelli, H., et al., Br. J. Pharmacol., 112, 127, 1994.
Barelli, H., et al., “Potent inhibition of endopeptidase 24.16 and endopeptidase 24.15 by the phosphornamide peptide N-(phenylethylphosphonyl)-Gly-L-Pro-L-aminohexanoic acid”, Biochem. J., 287 (2), 621-625, 1992.
Berge, S. M.: “Pharmaceutical salts”, J. Pharmaceutical Science, 66, 1-19 (1977).
Bickel, M. H.,: “The pharmacology and Biochemistry of N-oxides”, Pharmaco-logical Reviews, 21(4), 325-355, 1969.
Bundgaard, H. (editor), “Design of Prodrugs”, Elsevier, 1985.
Byrn et al., Pharmaceutical Research, 12(7), 945-954, 1995.
Dauch, P. et al., “Specific inhibition of endopeptidase 24.16 by dipeptides”, Eur. J. Biochem., vol. 202, pp. 269-276, 1991^{a}
Dauch, P. et al., “Fluorimetric assay of the neurotensin-degrading metallo-endopeptidase, endopeptidase24.16”, Biochem. J., 280, 1991, pp. 421-426. 1991^{b}
Dwyer & Meilor,: “Chelating agents and Metal Chelates”, Academic Press, chapter 7, 1964.
Ettmayer, P. et al., “Lessons learned from marketed and investigational prodrugs”, J. Med. Chem., 47, 2393-2404, 2004.
Järvinen, T. et al., “Design and Pharmaceutical applications of prodrugs”, pages 733-796 in: S. C. Gad (editor): “Drug Discovery Handbook”, John Wiley & Sons Inc., New Jersey, U.S.A., 2005.
Kaplan et al., Biochemistry, 30, 8165-8170, 1991.
Karanewsky et al., J. Med. Chem., 31, 204-212, 1988.
King, F. D., (editor), page 215 in: “Medicinal Chemistry: Principles and Practice”, 1994, ISBN 0-85186-494-5.
Martin, E. W. (Editor), “Remington: The Science and Practice of Pharmacy”, Mack Publishing Company, 19^{th} Edition, Easton, Pa., Vol 2., Chapter 83, 1447-1462, 1995.
Orlowski et al, Biochemistry, vol.27, pp. 597-602, 1988.
Stella, J., “Prodrugs as therapeutics”, Expert Opin. Ther. Patents, 14(3), 277-280, 2004.
Vincent, B. et al., “Phosphorous containing peptides as mixed inhibitors of endopeptidase 3.4.24.15 and 3.4.24.16: effect on neurotensin degradation in vitro and in vivo”, Br. J. Pharmacol., 115(6), 1053-1063, 1995.
Yiotakis, A. et al., “Phosphinic peptide analogues as potent inhibitors of Corynebacterium rathayii bacterial collagenase”, Biochem J., 303, 323-327, 1994.

### PATENTS AND PATENT APPLICATIONS

EP 0 071 544
EP 0 565 450
EP 0 725 075
FR-A-2 654 430
FR-A-2 676 059
U.S. Pat. No. 4,594,199
U.S. Pat. No. 4,602,092.
WO 97/048409
WO 98/03516.

What is claimed is:

1. A compound of formula (1), 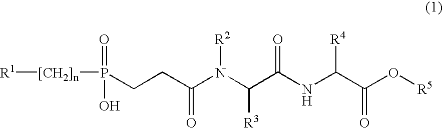
or a tautomer, a stereoisomer, an N-oxide, an isotopically-labeled analogue, or a pharmacologically acceptable salt, hydrate or solvate of any of the foregoing, wherein
R¹ is chosen from a monocyclic aryl group, a monocyclic heteroaryl group, a bicyclic aryl group and a bicyclic heteroaryl group, which groups are optionally substituted;
when R¹ is a monocyclic aryl group or a monocyclic heteroaryl group, n is 3, 4, or 5, and when R¹ is a bicyclic aryl group or a bicyclic heteroaryl group, n is 1, 2, 3, 4 or 5;
R² is a hydrogen atom or a (C₁₋₃)alkyl group, or R² and R³, together with the atoms to which they are attached, may form a five or six membered ring, which may contain a sulfur atom;
R³ is chosen from a hydrogen atom, a branched or unbranched (C₁₋₈)alkyl group, and an optionally substituted benzyl group;
R⁴ is chosen from a hydrogen atom, a branched or unbranched (C₁₋₈)alkyl group, and an optionally substituted benzyl group; and
R⁵ is chosen from a hydrogen atom, a methyl group, an ethyl group, a methoxymethyl group, and an ethoxymethyl group.

2. The compound as claimed in claim 1, wherein R¹ is an optionally substituted phenyl or naphthyl group, R² is a hydrogen atom or a methyl group, or R² and R³, together with the atoms to which they are attached, may form a five-membered ring, which may contain a sulfur atom.

3. The compound as claimed in claim 1, wherein R¹ is a phenyl or naphthyl group, R² is a hydrogen atom, or R² and R³, together with the atoms to which they are attached, may form a five-membered ring, which may contain a sulfur atom, R³ is a branched or unbranched (C₁₋₄)-alkyl group, R⁴ is a branched or unbranched (C₁₋₄)alkyl group, and R⁵ is a hydrogen atom.

4. The compound as claimed in claim 1, wherein the compound is an optically active enantiomer.

5. The compound as claimed in claim 1, wherein the compound is a compound of formula (1′): 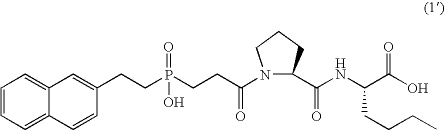
or a tautomer, a stereoisomer, an N-oxide, an isotopically-labeled analogue, or a pharmacologically acceptable salt, hydrate or solvate of any of the foregoing.

6. A compound of formula (2): 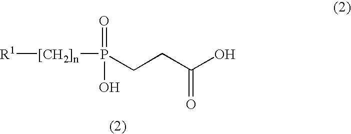
wherein
R¹ is chosen from a monocyclic aryl group, a monocyclic heteroaryl group, a bicyclic aryl group, and a bicyclic heteroaryl group, which groups are optionally substituted;
when R¹ is a monocyclic aryl group or a monocyclic heteroaryl group, n is 3, 4 or 5, and when R¹ is a bicyclic aryl group or a bicyclic heteroaryl group, n is 1, 2, 3, 4 or 5, with the proviso that when n is 4, R¹ is not an unsubstituted phenyl group.

7. A medicament comprising a compound of formula (1), 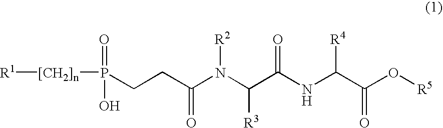
or a tautomer, a stereoisomer, an N-oxide, an isotopically-labeled analogue, or a pharmacologically acceptable salt, hydrate or solvate of any of the foregoing, wherein
R¹ is chosen from a monocyclic aryl group, a monocyclic heteroaryl group, a bicyclic aryl group and a bicyclic heteroaryl group, which groups are optionally substituted;
when R¹ is a monocyclic aryl group or a monocyclic heteroaryl group, n is 3, 4, or 5, and when R¹ is a bicyclic aryl group or a bicyclic heteroaryl group, n is 1, 2, 3, 4 or 5;
R² is a hydrogen atom or a (C₁₋₃)alkyl group, or R² and R³, together with the atoms to which they are attached, may form a five or six membered ring, which may contain a sulfur atom;
R³ is chosen from a hydrogen atom, a branched or unbranched (C₁₋₈)alkyl group, and an optionally substituted benzyl group;
R⁴ is chosen from a hydrogen atom, a branched or unbranched (C₁₋₈)alkyl group, and an optionally substituted benzyl group; and
R⁵ is chosen from a hydrogen atom, a methyl group, an ethyl group, a methoxymethyl group, and an ethoxymethyl group.

8. The medicament as claimed in claim 7, wherein R¹ is an optionally substituted phenyl or naphthyl group, R² is a hydrogen atom or a methyl group, or R² and R³, together with the atoms to which they are attached, may form a five-membered ring, which may contain a sulfur atom.

9. The medicament as claimed in claim 7, wherein R¹ is a phenyl or naphthyl group, R² is a hydrogen atom, or R² and R³, together with the atoms to which they are attached, may form a five-membered ring, which may contain a sulfur atom, R³ is a branched or unbranched (C₁₋₄)-alkyl group, R⁴ is a branched or unbranched (C₁₋₄)alkyl group, and R⁵ is a hydrogen atom.

10. A pharmaceutical composition comprising, at least one pharmaceutically acceptable carrier, at least one pharmaceutically acceptable auxiliary substance, or a combination of two or more thereof; and a therapeutically effective amount of at least one compound of formula (1), 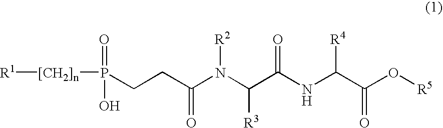
or a tautomer, a stereoisomer, an N-oxide, an isotopically-labeled analogue, or a pharmacologically acceptable salt, hydrate or solvate of any of the foregoing, wherein
R¹ is chosen from a monocyclic aryl group, a monocyclic heteroaryl group, a bicyclic aryl group and a bicyclic heteroaryl group, which groups are optionally substituted;
when R¹ is a monocyclic aryl group or a monocyclic heteroaryl group, n is 3, 4, or 5, and when R¹ is a bicyclic aryl group or a bicyclic heteroaryl group, n is 1, 2, 3, 4 or 5;
R² is a hydrogen atom or a (C₁₋₃)alkyl group, or R² and R³, together with the atoms to which they are attached, may form a five or six membered ring, which may contain a sulfur atom;
R³ is chosen from a hydrogen atom, a branched or unbranched (C₁₋₈)alkyl group, and an optionally substituted benzyl group;
R⁴ is chosen from a hydrogen atom, a branched or unbranched (C₁₋₈)alkyl group, and an optionally substituted benzyl group; and
R⁵ is chosen from a hydrogen atom, a methyl group, an ethyl group, a methoxymethyl group, and an ethoxymethyl group.

11. The pharmaceutical composition as claimed in claim 10, wherein the composition further comprises at least one additional therapeutic agent.

12. The pharmaceutical composition as claimed in claim 10, wherein R¹ is an optionally substituted phenyl or naphthyl group, R² is a hydrogen atom or a methyl group, or R² and R³, together with the atoms to which they are attached, may form a five-membered ring, which may contain a sulfur atom.

13. The pharmaceutical composition as claimed in claim 10, wherein R¹ is a phenyl or naphthyl group, R² is a hydrogen atom, or R² and R³, together with the atoms to which they are attached, may form a five-membered ring, which may contain a sulfur atom, R³ is a branched or unbranched (C₁₋₄)-alkyl group, R⁴ is a branched or unbranched (C₁₋₄)alkyl group, and R⁵ is a hydrogen atom.

14. A method for regulating blood pressure, or gastric emptying, or treating Parkinson's disease, anxiety, depression, or psychosis, the method comprising administering a therapeutically effective amount of a compound of formula (1), 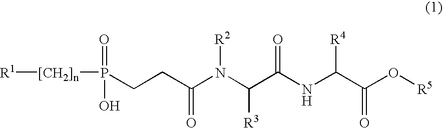
or a tautomer, a stereoisomer, an N-oxide, an isotopically-labeled analogue, or a pharmacologically acceptable salt, hydrate or solvate of any of the foregoing, wherein
R¹ is chosen from a monocyclic aryl group, a monocyclic heteroaryl group, a bicyclic aryl group and a bicyclic heteroaryl group, which groups are optionally substituted;
when R¹ is a monocyclic aryl group or a monocyclic heteroaryl group, n is 3, 4, or 5, and when R¹ is a bicyclic aryl group or a bicyclic heteroaryl group, n is 1, 2, 3, 4 or 5;
R² is a hydrogen atom or a (C₁₋₃)alkyl group, or R² and R³, together with the atoms to which they are attached, may form a five or six membered ring, which may contain a sulfur atom;
R³ is chosen from a hydrogen atom, a branched or unbranched (C₁₋₈)alkyl group, and an optionally substituted benzyl group;
R⁴ is chosen from a hydrogen atom, a branched or unbranched (C₁₋₈)alkyl group, and an optionally substituted benzyl group; and
R⁵ is chosen from a hydrogen atom, a methyl group, an ethyl group, a methoxymethyl group, and an ethoxymethyl group,
to a human or animal patient in need of such treating.

15. The method as claimed in claim 14, wherein R¹ is an optionally substituted phenyl or naphthyl group, R² is a hydrogen atom or a methyl group, or R² and R³, together with the atoms to which they are attached, may form a five-membered ring, which may contain a sulfur atom.

16. The method as claimed in claim 14, wherein R¹ is a phenyl or naphthyl group, R² is a hydrogen atom, or R² and R³, together with the atoms to which they are attached, may form a five-membered ring, which may contain a sulfur atom, R³ is a branched or unbranched (C₁₋₄)-alkyl group, R⁴ is a branched or unbranched (C₁₋₄)alkyl group, and R⁵ is a hydrogen atom.

17. The method as claimed in claim 14, wherein the method further comprises administering an additional therapeutic agent prior to, simultaneously with, or following the administration of the compound of formula (1) or a tautomer, a stereoisomer, an N-oxide, an isotopically-labeled analogue, or a pharmacologically acceptable salt, hydrate or solvate of any of the foregoing, to the human or animal patient in need of such treating.

18. A method of inhibiting neurotensin degrading enzymes, the method comprising administering a therapeutically effective amount of a compound of formula (1), 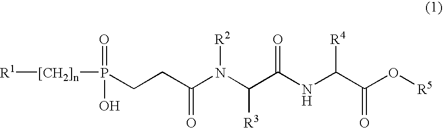
or a tautomer, a stereoisomer, an N-oxide, an isotopically-labeled analogue, or a pharmacologically acceptable salt, hydrate or solvate of any of the foregoing, wherein
R¹ is chosen from a monocyclic aryl group, a monocyclic heteroaryl group, a bicyclic aryl group and a bicyclic heteroaryl group, which groups are optionally substituted;
when R¹ is a monocyclic aryl group or a monocyclic heteroaryl group, n is 3, 4, or 5, and when R¹ is a bicyclic aryl group or a bicyclic heteroaryl group, n is 1, 2, 3, 4 or 5;
R² is a hydrogen atom or a (C₁₋₃)alkyl group, or R² and R³, together with the atoms to which they are attached, may form a five or six membered ring, which may contain a sulfur atom;
R³ is chosen from a hydrogen atom, a branched or unbranched (C₁₋₈)alkyl group, and an optionally substituted benzyl group;
R⁴ is chosen from a hydrogen atom, a branched or unbranched (C₁₋₈)alkyl group, and an optionally substituted benzyl group; and
R⁵ is chosen from a hydrogen atom, a methyl group, an ethyl group, a methoxymethyl group, and an ethoxymethyl group,
to a patient in need thereof.

19. The method as claimed in claim 18, wherein R¹ is an optionally substituted phenyl or naphthyl group, R² is a hydrogen atom or a methyl group, or R² and R³, together with the atoms to which they are attached, may form a five-membered ring, which may contain a sulfur atom.

20. The method as claimed in claim 18, wherein R¹ is a phenyl or naphthyl group, R² is a hydrogen atom, or R² and R³, together with the atoms to which they are attached, may form a five-membered ring, which may contain a sulfur atom, R³ is a branched or unbranched (C₁₋₄)-alkyl group, R⁴ is a branched or unbranched (C₁₋₄)alkyl group, and R⁵ is a hydrogen atom.

21. The method as claimed in claim 18, wherein the method further comprises administering an additional therapeutic agent prior to, simultaneously with, or following the administration of the compound of formula (1) or a tautomer, a stereoisomer, an N-oxide, an isotopically-labeled analogue, or a pharmacologically acceptable salt, hydrate or solvate of any of the foregoing, to the human or animal patient in need thereof.

22. A process for preparing a pharmaceutical composition comprising:
i) combining a compound of formula (1) 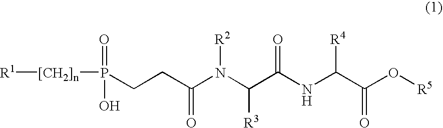
or a tautomer, a stereoisomer, an N-oxide, an isotopically-labeled analogue, or a pharmacologically acceptable salt, hydrate or solvate of any of the foregoing, with at least one pharmaceutically acceptable adjuvant, diluent or carrier, wherein
R¹ is chosen from a monocyclic aryl group, a monocyclic heteroaryl group, a bicyclic aryl group and a bicyclic heteroaryl group, which groups are optionally substituted;
when R¹ is a monocyclic aryl group or a monocyclic heteroaryl group, n is 3, 4, or 5, and when R¹ is a bicyclic aryl group or a bicyclic heteroaryl group, n is 1, 2, 3, 4 or 5;
R² is a hydrogen atom or a (C₁₋₃)alkyl group, or R² and R³, together with the atoms to which they are attached, may form a five or six membered ring, which may contain a sulfur atom;
R³ is chosen from a hydrogen atom, a branched or unbranched (C₁₋₈)alkyl group, and an optionally substituted benzyl group;
R⁴ is chosen from a hydrogen atom, a branched or unbranched (C₁₋₈)alkyl group, and an optionally substituted benzyl group; and
R⁵ is chosen from a hydrogen atom, a methyl group, an ethyl group, a methoxymethyl group, and an ethoxymethyl group; and
ii) formulating the combination produced in (i) into a suitable dosage form.

23. The process as claimed in claim 22, wherein R¹ is an optionally substituted phenyl or naphthyl group, R² is a hydrogen atom or a methyl group, or R² and R³, together with the atoms to which they are attached, may form a five-membered ring, which may contain a sulfur atom.

24. The process as claimed in claim 22, wherein R¹ is a phenyl or naphthyl group, R² is a hydrogen atom, or R² and R³, together with the atoms to which they are attached, may form a five-membered ring, which may contain a sulfur atom, R³ is a branched or unbranched (C₁₋₄)-alkyl group, R⁴ is a branched or unbranched (C₁₋₄)alkyl group, and R⁵ is a hydrogen atom.

25. The process as claimed in claim 22, wherein the combination of step (i) further comprises an additional therapeutic agent.
